(19) United States
(12) Patent Application Publication
(10) Pub. No.: US 2026/0345312 A1
(43) Pub. Date: Nov. 5, 2026

(54) IMPLANTABLE 3D BRAIDED ENDOVASCULAR PROSTHESIS FOR REMODELING OF DISSECTED AORTA

(71) Applicant: INTRESSA VASCULAR S.A., ISNES (BE)

(72) Inventors: Kanae DODA, Hastière-par-Dela (BE); Loic DREANO, Lyon (FR); Diane LEJEUNE, BRUSSELS (BE); Pierre DOUETTE, EMBOURG (BE)

(21) Appl. No.: 19/099,237

(22) PCT Filed: Jul. 25, 2023
(86) PCT No.: PCT/EP2023/070609

§ 371 Date: Jan. 28, 2025

(30) Foreign Application Priority Data
Jul. 29, 2022 (EP) 22187697.2

(51) Int. Cl. A61F 2/07

(52) U.S. Cl. CPC A61F 2/07; A61F 2230/0069

(57) ABSTRACT

The disclosure relates to implantable endovascular prostheses for use in the treatment of aortic dissection. The endovascular prostheses comprises at least one self-expandable braided framework extending along an axis able to expand from a radially compressed state in a delivery configuration to a radially expanded state. The self-expandable braided framework is formed by wires of a same diameter, devoid of any impermeable cover layer, comprising a plurality of layers of wires made of biocompatible material. The braid framework forms a part of the wall of the implantable endovascular prosthesis. The plurality of layers of wires consists of a lumen comprised in the self-expandable braided framework, forming a cylindrical shape with a circular cross-section. Each layer forms a mesh. The meshes are interlocked and form a lattice with a plurality of wires of said layers. The wires are integrated in the mesh of at least one of the adjacent layers.

### CROSS-REFERENCE TO RELATED APPLICATION(S)

This present application is a national stage application of International Patent Application No. PCT/EP2023/070609, filed Jul. 25, 2023, which claims priority to European Patent Application No. 22187697.2, filed Jul. 29, 2022, the disclosures of which are hereby incorporated by reference in their entireties.

### TECHNICAL FIELD

The present disclosure relates to implantable endovascular prostheses for treatment of aortic dissection.

### BACKGROUND

The aorta is the largest blood vessel in the body and responsible for transporting oxygen rich blood from the heart to the rest of body. It is shaped like a candy cane, with the first section moving up towards the head (ascending aorta), then curving in a C-shape as smaller arteries that stem therefrom carry blood to the head and arms (aortic arch). After the curve, the aorta becomes straight again, and moves downward towards the abdomen, carrying blood to the lower part of the body (descending aorta). The walls of the aorta consist of three layers that ensure structural support and morphology.

Aortic dissection is one of the aortic catastrophes with a high mortality and morbidity. It typically occurs when the inner layer of the artery's wall weakens and a primary tear occurs into the middle layer of the wall. When this happens, blood can pass through the tear, causing the layers to separate from one another, or dissect. If left untreated, the tear can enlarge. This leads to the formation of a new channel, called a false lumen (FL), separate from the true lumen (TL) by an intimal flap. This FL can progressively extend from the tear to the lowest part of the aorta, preventing blood from flowing properly in the TL to irrigate end-organs branched onto the aorta.

As shown in FIG. 1, the separation of the inner layer of the aorta which forms the intimal flap 104 can have multiple entry holes, known as re-entry tears 101. These secondary tears allow blood to flow between the TL 100 and the FL 102. Over time, the FL will compress the true lumen, hence reduce the flow of blood going to the end-organs leading to ischemia. Sometimes, the blood breaks through the outer layer of the aorta, a complication called aortic rupture, causing a life-threatening loss of blood and drop in blood pressure that requires immediate surgery. Aortic dissections that occur in the ascending part of the aorta are called Stanford type A dissections; those in the descending aorta are Stanford type B dissections. Type B dissections are also classified based on chronicity into hyperacute, acute, subacute and chronic wherein the hyperacute phase corresponds to the 24 h first hours from the onset of the symptom, the acute phase as the time between 24 h and 14 days of the onset of symptoms, subacute between 2 weeks and 3 months and chronic after 3 months.

Traditionally, type B aortic dissections (TBAD) which are uncomplicated at presentation, are treated with optimal medical therapy, and lifestyle modification to minimize risk of progression. However, up to 50% of patients may suffer aorta-related complication such as FL enlargement or rupture and retrograde dissection. If imaging shows such complications, repair should be considered. Besides, evidence is accumulating that dissections presenting as complicated (defined as rupture or with clinical signs of malperfusion) or high-risk (defined as associated with refractory pain, refractory hypertension, bloody pleural effusion, aortic diameter >40 mm, radiographic only malperfusion, readmission, an entry tear located in the lesser curve of the aorta, or a false lumen diameter >22 mm) should be treated with endovascular repair.

Indeed, stent graft endovascular repair is known as alternative option to conventional open surgical repair. It is expected to promote aortic remodeling with favorable FL regression and favorable TL expansion. The limitations of stent grafts are linked to their intrinsic natures and characteristics. First, due to the impermeable character of the graft material, placing a stent graft in front of side branches will occlude such branches and lead to undesired end-organ ischemia (e.g., spinal cord injury, kidney failure). Short stent grafts are therefore often used to limit such side effects, but come with the downside that they are not able to cover the whole dissected aorta. This can lead to continued retrograde FL perfusion with back-flow from secondary entry tears located distally from the stent graft and/or extension of dissection beyond the treated area, in turn leading to reintervention.

Additionally, incidents of late distal stent graft-induced new entry (SINE) and late extensions of aortic dissection are often reported involving the thoracic as well as the abdominal and iliac portions. It may be caused by high radial force and stiffness of stent-graft partially covering the dissected portion of aorta and pulsatile motion that erode the fragile intima at the distal edge of the stent graft.

In order to overcome these challenging problems, endovascular treatment strategies should not only target the occlusion of the proximal entry-tear but should focus in addition on the prevention of FL back-flow by providing extended mechanical support to the dissected aorta as to promote sufficient aortic remodeling.

Accordingly, there is a need for an implantable endovascular prosthesis having mechanical properties suitable to achieve favorable aortic remodeling, and allowing to cover long segments of the aorta with more entry points to reduce pressure and flow transmission into the FL, while maintaining the blood flow in branches of which the orifices are covered by said endovascular prosthesis at the thoraco-abdominal level without significant risk of morbidity such as spinal cord ischemia.

U.S. Patent Application No. 2004/0073293 discloses a three-dimensionally braided structure composed of metal wires and textile strands. This structure is designed to create an impermeable device for the treatment of aneurysms. By utilizing the impermeable body, the device effectively blocks the blood flow into the aneurysmal sac, while also exhibiting enhanced resistance to radial compression due to its three-dimensionally braided stent. However, the impermeable body of the device does not address the issue of preventing occlusion of side branches.

U.S. Patent Application No. 2004/0215332 discloses a multilayered braided bare stent used for aneurysm treatment. It mentions that the multilayer structure increases resistance to crushing without reducing flexibility, but it does not specify the required braid pattern to exhibit these properties.

U.S. Pat. No. 11,540,930 also discloses a multilayered braided bare stent used for aneurysm treatment. The stent has a device thickness (T) that is over 3 times the wire diameter (D). This type of stent can effectively form a thrombus in the aneurysmal sac while maintaining side branch perfusion. However, the patent does not mention any specific braid pattern as a technical feature of the stent to obtain mechanical properties suitable for achieving favorable aortic remodeling in patients with aortic dissection.

### SUMMARY

An object of the present disclosure is to provide a device implantable by endovascular approach for treating aortic dissection.

The subject of the disclosure is defined in the appended independent claims. Preferred embodiments are defined in the dependent claims.

A subject of the present disclosure is an implantable endovascular prosthesis comprising at least one self-expandable braided framework extending along an axis able to expand from a radially compressed state in a delivery configuration to a radially expanded state. Said braided framework is formed by wires of a same diameter D₃ and devoid of any impermeable cover layer. The braided framework comprises a plurality of layers of wires made of biocompatible material and forms a part of the wall of the implantable endovascular prosthesis. Said plurality of layers of wires consists of a lumen comprised in the braided framework. It forms a cylindrical shape with a circular cross-section. Each of said layers forms a mesh. Said meshes form a lattice with a plurality of wires of the layers. The meshes being interlocked. The wires are integrated in the mesh of at least one of the adjacent layers.

Each of the wires forms a helical path in a direction or in the other around the axis of the self-expandable braided framework while crossing with wires of the opposite helical direction being positioning at either the outer or the inner side of the braided framework. A ratio T₂/D₃ of a thickness T₂ of a wall of said braided framework to the diameter D₃ of the wires is at least 2.8, preferably at least 3.0, most preferably at least 3.5. Wires taking position at the outer side the most often among wires of the same helical direction when crossing with wires of the opposite helical direction during a complete helical wrap are defined as wires belonging to the outermost layer of the braided framework (OM wires). Wires taking position at the inner side the most often among wires of the same helical direction when crossing with wires of the opposite helical direction during a complete helical wrap being defined as wires belonging to the innermost layer of the braided framework (IM wires). The number of OM wires is at least 5% and at most 35% of the total number of wires forming the braided framework, the number of IM wires being at least 10% and at most 35% of the total number wires forming the braided framework.

A pattern indicating positions of wires at crossing points is preferably repeating at least 4 times within a complete helical wrap of the wires in both helical directions, more preferably at least 8 times.

The number of wires forming said braided framework but neither being OM wire nor being IM wire is preferably at least 50% and at most 70% of the total number of wires forming the braided framework.

A ratio of the number of times that an OM wire crosses at outer side to the number of the total crosses made within its complete helical wrap is preferably at least 70% and at most 90%.

The number of wires forming the braided framework is preferably at least 72, more preferably at least 96, even more preferably at least 120 and 200, still even more preferably between 150 and 180.

A surface coverage ratio (SCR) of the braided framework in the fully expanded state is preferably comprised between 25% and 45%, more preferably between 30 and 40%.

The wires forming the braided framework have preferably a diameter D₃ of at least 120 μm and at most 250 μm, more preferably at least 150 μm and at most 220 μm, even more preferably at least 180 μm and at most 210 μm.

In a preferable embodiment, the cylindrical form of the braided framework has preferably a constant diameter in the fully expanded state. Preferably the braided framework has an inner diameter of at least 20 mm and at most 50 mm in the fully expanded state.

In another preferable embodiment, the braided framework has larger diameter one or both end(s) in the fully expanded state. Preferably an inner diameter of the braided framework is at least 20 mm and at most 50 mm in the fully expanded state.

### BRIEF DESCRIPTION OF THE FIGURES

FIG. 1 is a diagrammatic view of Type B aortic dissection.

FIG. 2 is a schematic front view of a self-expandable braided framework according to the present disclosure in a preferable embodiment.

FIG. 2a is a schematic magnified view of a portion of the front view shown in FIG. 2.

FIG. 3 is a side view of the self-expandable braided framework shown in FIG. 2.

FIG. 4 is a section view of the self-expandable braided framework shown in FIGS. 2, 2a and 3 according to a cutting plane IV-IV.

FIG. 4a shows is a schematic magnified view of a portion of the cross-section shown in FIG. 4.

FIG. 5 is a schematic magnified view of a wall of a self-expandable braided framework according to present disclosure.

FIG. 6 is a magnified image of a self-expandable braided framework according to present disclosure.

FIG. 6a is a magnified image of FIG. 6.

FIGS. 7a to 7c are computed tomography (CT) scans of a clinical case before and after treated by an implantable endovascular prosthesis according to present disclosure.

FIGS. 8a to 8c are computed 3D fusion images constructed based on CT scans of the clinical case shown in FIGS. 7a to 7c.

FIGS. 9a to 9c are computed tomography (CT) scans of another clinical case before the procedure, at discharge, and at the 68-month follow-up, treated using an implantable endovascular prosthesis according to the present disclosure.

FIGS. 10a to 10c are computed 3D fusion images constructed based on CT scans of the clinical case shown in FIGS. 9a to 9c.

FIG. 11 is a section of braiding guide path described in EP1248872.

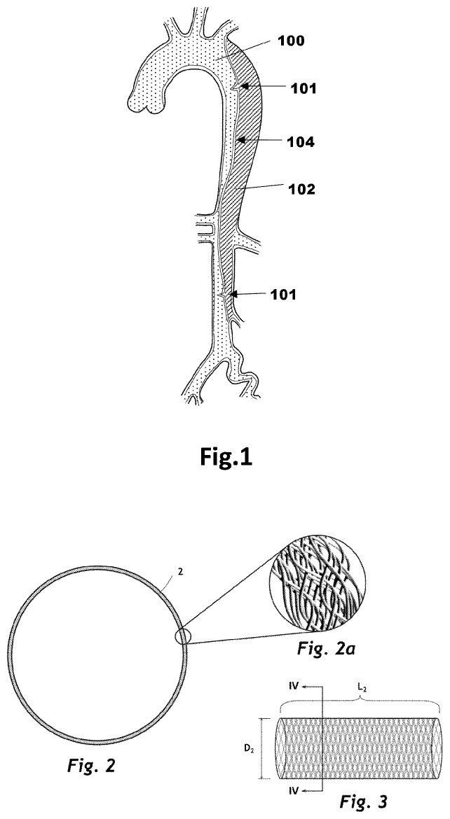

### DETAILED DESCRIPTION

As used hereinafter, the term “implantable” refers to an ability of a medical device to be positioned at a location within a body vessel through invasive means. Implantable medical device can be configured for transient placement within a body vessel during a medical intervention (e.g., seconds, minutes, hours), or to remain in a body vessel permanently.

The terms “endovascular” prosthesis refers to a device adapted for invasive placement in a curved or straight body vessel by procedures wherein the prosthesis is advanced within and through the lumen of a body vessel from a remote location to a target site within the body vessel. In vascular procedures, a medical device can typically be introduced “endovascularly” using a catheter over a guide wire under fluoroscopic guidance. The catheters and wire guides may be introduced through conventional access sites in the vascular system.

The term “catheter” refers to a tube that is inserted into a blood vessel to access the target site. In the present description, a “catheter” will designate either a catheter per se, or a catheter with its accessories, meaning needle, guide wire, introducer sheath and other common suitable medical devices known by the man skilled in the art.

The term “permanent” refers to a medical device which may be placed in a blood vessel and will remain in the blood vessel for a long period of time (e.g., months, years) and possibly for the remainder of the patient's life.

The terms “expanded shape” or “expanded state” refer to a shape or state resulting from the self-expanding properties of a self-spring-back object when it is allowed to expand without any outer compression force (i.e., non-constricted state).

The implantable endovascular prosthesis 1 according to the present disclosure comprises at least one self-expandable braided framework 2. The self-expandable braided framework 2 is configured to take a compressed shape having a relatively small and relatively uniform diameter when disposed within a delivery system (i.e., “in compressed state”), and to spontaneously take a deployed shape with radially expanded diameter within the delivery location such as a body lumen (i.e., “in deployed state”). The self-expandable braided framework 2 has a 3D braided structure having a plurality of interlocked layers (interlocked multilayer configuration) formed by braiding a plurality of wires. The self-expandable braided framework 2 comprises a lumen in a cylindrical form with a circular cross-section shown in FIGS. 2, 2a and 3.

In a preferable embodiment, the implantable endovascular prosthesis 1 can substantially consist of the self-expandable braided framework 2.

FIG. 4 shows a schematic cross-section of the implantable endovascular prosthesis 1 according to the present disclosure. FIG. 4a shows a schematic magnified view of a portion of the implantable endovascular prosthesis 1 consisting of a self-expandable framework 2.

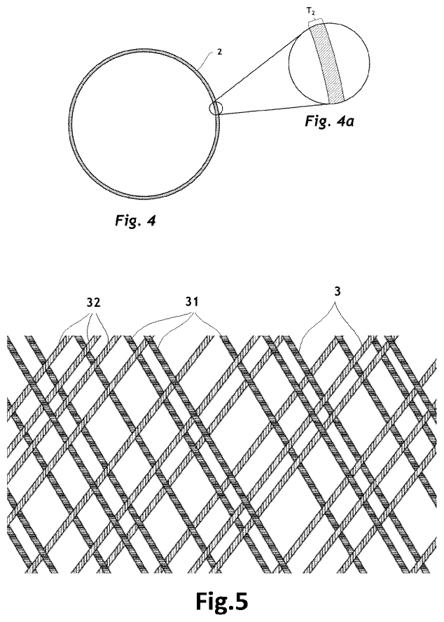

The self-expandable braided framework 2 has a thickness T₂ in a fully expanded state. It may be measured by using a Micro-CT scan technology or a digital optical comparator to identify a circumscribed circle and Inscribed circle of a cross section of the braided framework 2 and to divide by 2 the difference between the diameters of these circles. The term “interlocked multilayer” refers to a framework comprising multiple layers, whose plies are not distinct at the time of braiding, for example a given number of wires of the plies of the first layer being interlocked with the plies of the second layer and/or other layers as schematically illustrated in FIG. 5. Said interlocked multilayer configuration, for example, can be provided by using the braiding machine described in EP1248872. When the self-expandable braided framework 2 is observed perpendicularly with respect to its wall, meshes of the self-expandable braided framework 2 forms lattices with a plurality of level of wires 3.

A ratio T₂/D₃ of the thickness T₂ of a wall of the self-expandable braided framework 2 to the diameter D₃ of wire 3 forming of the braided framework 2 should be greater than 2.0. It characterizes that the self-expandable braided framework 2 has more than a single layer of mesh, namely 3D braided structure with a multilayered configuration. The ratio T₂/D₃ is preferably at least 2.8, more preferably at least 3.0, even more preferably at least 3.5. The greater the ratio T₂/D₃, the greater in the degree of multilayering. The thickness T₂ of the self-expandable braided framework 2 may vary depending on factors such as the braiding pattern, the diameter of the braided framework, and the properties of the wires. As a result, the thickness T₂ is not systematically equal to the number of layers multiplied by the wire diameter (D₃).

The term “OM wire” refers to a wire belonging to the outermost layer of the self-expandable braided framework 2, which can be defined as a wire taking position at the outer side the most often among wires of the same helical direction when crossing with wires of the opposite helical direction during its complete helical wrap when the crossings of wires 3 were observed perpendicular to the wall of the braid framework 3. By most often, it is meant preferably at least 70% of the total number of crossings on a complete helical wrap. FIG. 5 shows wires 31 in the same helical direction and wires 32 in the opposite helical direction as to wires 31. The term “IM wire” means a wire belonging to the innermost layer of the self-expandable braided framework 2, which can be defined as a wire taking position at the inner side the most often among wires of the same helical direction when crossing with wires of the opposite helical direction during its complete helical wrap. The positions of the wires at crossing points can be identified by observing perpendicularly with respect to a wall of the self-expandable braided framework 2. Namely, the term “position at the outer side” means a wire crossing over another wire, and the term “position at the inner side” meaning a wire crossing down another wire when a crossing point was observed perpendicularly with respect to the wall. In a preferable embodiment, the number of OM wires included in the self-expandable braided framework 2 is at least 5% and at most 35% in the total number of wires 3 forming of the braided framework 2; and the number of IM wires being at least 10% and at most 35% in the total number wires 3.

The number of times that “OM wire” is positioned at outer side at crosses during its helical wrap is preferably at most 90%, more preferably at least 80%. The number of times that “IM wire” is positioned at outer side at crosses during its helical wrap is preferably at least 5% and at most 20%, more preferably at least 10%. A pattern indicating such positions of wires at crossing points is preferably repeating at least 4 times within a complete helical wrap of the wires in both helical directions, more preferably at least 8 times. Wires not belonging to the outmost layer nor the innermost layer can be defined as wires belonging to middle layers. The number of wires belonging to middle layers is preferably at least 50% and at most 70% in the total number of wires forming the braided framework. The more the number of times OM wire positioning at outer side, the more the implantable endovascular prosthesis being flexible while maintaining the same radial force for progressive remodeling of dissected aorta.

The surface coverage ratio (SCR) of self-expandable braided framework 2 is preferably between 25% and 45%, more preferably between 30% and 40% in fully expanded state. The SCR of the endoluminal prosthesis is defined by the relation:

SCR=Sw/St

Wherein “Sw” is the actual surface covered by wires 3 composed in the braided framework 2, and “St” is the total surface of the wall of the braided framework 2 when observed normally with respect to the wall.

The self-expandable braided framework 2 of the endovascular prosthesis 1 is made of at most 196 wires 3, preferably at least 72 wires at most 160 wires. The wires preferably have a diameter D₃ of at least 120 μm, preferably at least 150 μm, more preferably at least 180 μm, even more at least 200 μm and at most 220 μm.

The biocompatible material used in the disclosure is preferably a metallic substrate selected from a group consisting of stainless steels (e.g., 316, 316L or 304); nickel-titanium alloys including shape memory or superelastic types (e.g., nitinol, Nitinol-DFT®-Platinum); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; magnesium alloys; titanium alloys (e.g., TiC, TiN); tantalum alloys (e.g., TaC, TaN); L605. Said metallic substrate is preferably selected from the group consisting of titanium, nickel-titanium alloys such as nitinol and Nitinol-DFT®-Platinum, any type of stainless steels, or a cobalt-chromium-nickel alloys such as Phynox®.

One of the technical effects provided by the 3D braided structure of self-expandable braided framework 2 is that, the implantable endovascular prosthesis 1 exhibits the attribute of spontaneously deploying within TL along the curve of aorta without balloon molding, and of pushing a dissection flap so that TL will be enlarged with a concomitant decrease of the FL. Accordingly, the implantable endovascular prosthesis 1 has sufficient flexibility to conform with the curves of aorta as well as an adequate mechanical property for pushing back the collapsed wall of TL and repositioning it progressively to its original non-dissected diameter.

In addition, thanks to the permeable property of the 3D braided structure of the self-expandable braided framework 2, the endovascular prosthesis 1 can keep the branches and collaterals unobstructed without additional repairs such as open debranching-bypass procedure and custom-made fenestrated/branched configuration for maintaining a blood flow are not required.

### Examples (3D Braided Configuration)

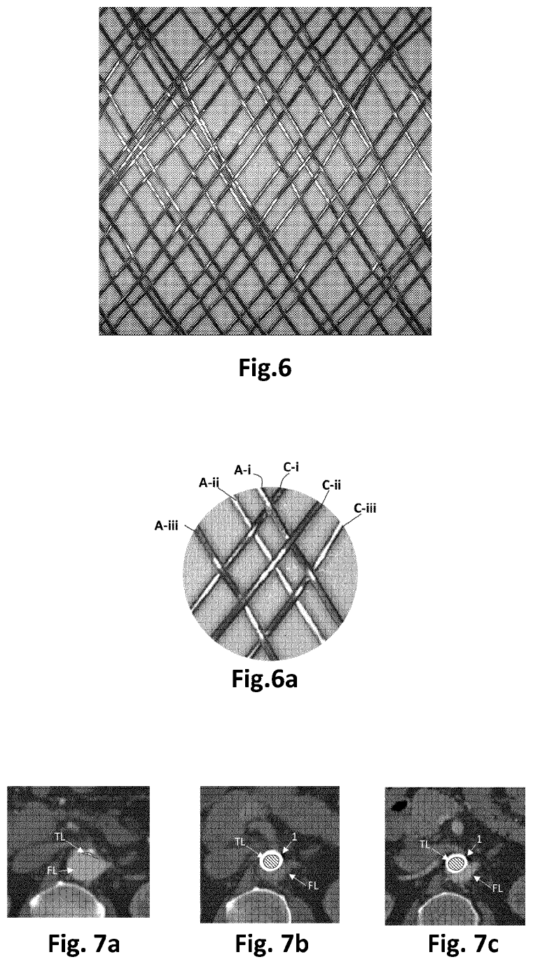

FIG. 6 shows a part of a self-expandable braided framework of Example 1 (Ex.1) according to the present disclosure. The braiding pattern of Ex. 1 was projected to

Table 1. The table shows which wires were positioned at outer side of the self-expandable braided frame at each crossing point within a repeating unit. For example, when a wire helically rotating in clockwise (C01) was positioning at outer side on a crossing point with a wire in anticlockwise (A01), a letter “C” was written in a cell of C01-A01. For example, to illustrate how to interpret the wire positions at crossing points, the braiding pattern shown in FIG. 6a was transposed into Table 2. In this table, wires C-i, C-ii, and C-iii were designated as braided in a clockwise direction (C-wires), while wires A-i, A-ii, and A-iii were designated as braided in an anticlockwise direction (A-wires). Ex. 1 was formed by 104 wires including 52 wires braided in clockwise (C-wires) and 52 wires in anticlockwise (A-wires). In a complete helical wrap (pitch), a C-wire crossed 52 times with A-wires. Every 13 crossings, the patterns of wire positions were repeated. Therefore, the repeating unit consisted of 13 C-wires and 13 A-wires. It was repeated 4 times within the complete helical wrap. In the repeating unit, 4 out of 13 C-wires (C02, C05, C08, C11) and 2 out of 13 A-wires (A03, A09) were identified as OM wires in the repeating unit (23.1%). Therefore, in total 24 out of 104 wires forming the braided framework were identified as OM wires as these wires take position at outer side the most often (11 times out of 13) in a pitch among wires of the same helical direction as summarized in Table 4. The OM wires of both clockwise and anticlockwise rotations were positioned at outer side 11 times out of 13 crossing points (84.6%) within the repeating unit. The IM wires of both rotations were positioned twice at outer side out of 13 crossing points (15.4%) as shown in

Table 1. These numbers counted in a complete helical wrap (pitch) are summarized in Table 4. The number of times that the repeating unit was found in a complete helical wrap is summarized in Table 5.

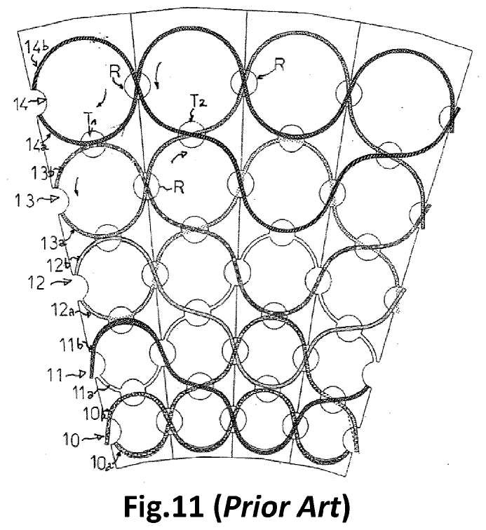

Ex.1 was braided using the multilayered braiding machine as described in EP1248872. Out of the 104 wires that consisted of the braided structure of Ex.1, 32 wires were allocated to pass through one of the guide paths along the outermost circumference (referred to as the OM group) such as the guide paths 13a, 14a, 14b in FIG. 11. Another set of 32 wires were designated to pass through one of the guide paths along the innermost circumference (referred to as the IM group) such as the guide paths 10a, 10b, 11b in FIG. 11. The remaining wires were designated to pass through guide paths that neither traversed the outermost nor innermost circumference (referred to as the Middle group, such as the guide paths 11a, 12a, 12b, 13b in FIG. 11). It is important to notice that FIG. 11 does not represent the used configuration, but just illustrates how the different wires paths are defined.

The thickness of braided framework Ex.1, measured using a digital optical comparator, was found to be 0.72 mm. Consequently, the T₂/D₃ ratio was calculated as 3.79 by dividing the measured thickness T₂ by the wire diameter D₃ of 190 μm as shown in Table 6.

Braiding and technical properties of other examples (Ex.2 and Ex.3) regarding self-expanded braided frameworks according to the present disclosures and comparative examples of Prior Art (CEx.1 and CEx.2) manufactured following the same procedure as Ex.1 (but with different path configuration) were summarized in Table 3 to Table 5 as well.

Thickness Measurement and T₂/D₃ Ratio:

The results of the thickness measurements and the calculated T₂/D₃ ratio, derived from the measured thickness and wire diameter, were summarized in Table 6. All braided frameworks, including Ex. 1 to Ex.3 and CEx. 1 and CEx.2, evaluated in this test exhibited a T₂/D₃ ratio exceeding 2.8.

### Crush Resistance and Elastic Recovery:

The purpose of the crush resistance test is to assess the stent ability to resist permanent deformation along the entire length of the device when subjected to a load uniformly applied over the length of the stent. It can quantify the energy required for the stent to go back to its initial state and determine if the stent recovers its original geometry after unloading (Elastic recovery).

The crush resistance of self-expandable braided frameworks according to present disclosure (Ex.1, Ex.2 and Ex.3), as well as the comparative samples of prior art (CEx.1 and CEx.2), were assessed by means of a compression resistance parallel plate test performed using a tensile tester (Universal Mechanical Testing System Lloyd, AMETEX, USA). These test samples were fully compressed between two flat plates with the upper plate advancing towards the lower one. Crush resistance was measured by recording the load and the associated displacement while compressing the samples up to full collapsing and while releasing the samples until the original position of the upper plate. Elastic recovery was calculated as the ratio between final and initial diameter, in percentage.

Crush resistance test results showed a clear difference in mechanical properties between the braided framework according to the present disclosure and prior art as shown in Table 7. There was a significant increase in resistance to crush force for one of the comparative samples of Prior Art (CEx.1) compared to the braided frameworks of the present disclosures but no increase for the other comparative sample (CEx.2). Elastic recovery was found to be at least 96% for the braided frameworks according to the present disclosure. Conversely, the elastic recovery rates for the comparative samples from the Prior Art, namely CEx.1 and CEx.2, were 87.5% and 93.3% respectively and both of which were inferior to the elastic recovery rates of the braided frameworks according to the present disclosure. It has been demonstrated that the braided frameworks, as per the present disclosure, exhibit a high elastic recovery property, providing ample flexibility to conform to the curves of the aorta after deployment. These frameworks are characterized by having a minimum of 5% and a maximum of 35% of the total number of wires as OM wires, and a minimum of 10% and a maximum of 35% of the total number of wires as IM wires.

### Self-Expandable Property (Conformability):

The self-expandable property of the braided frameworks was evaluated by visually observing their state after being released from a completely crushed state as found in the delivery catheter. The braided frameworks (Ex.1 to Ex.3) according to the present disclosure demonstrated excellent self-expanding ability, promptly returning to their pre-set diameter upon release from the crushed state in the delivery catheter without experiencing any significant deformation (Significant deformation is defined as an elastic recovery rate below 95%). In contrast, the comparative samples of the Prior Art (CEx.1 and CEx.2) exhibited a deformation under 95% in elastic recovery and significantly poor self-expandable ability as they could not be inserted completely into the delivery catheter of desired diameter suitable for endovascular treatment (i.e. as used in Ex.1 to Ex.3).

Based on the test results of elasticity recovery and self-expanding ability, the braided frameworks with braiding patterns according to the present disclosure exhibit high flexibility and conformability in comparison with the comparative samples. They enable favorable aortic remodeling while also providing coverage for long segments of the aorta.

### Clinical Results:

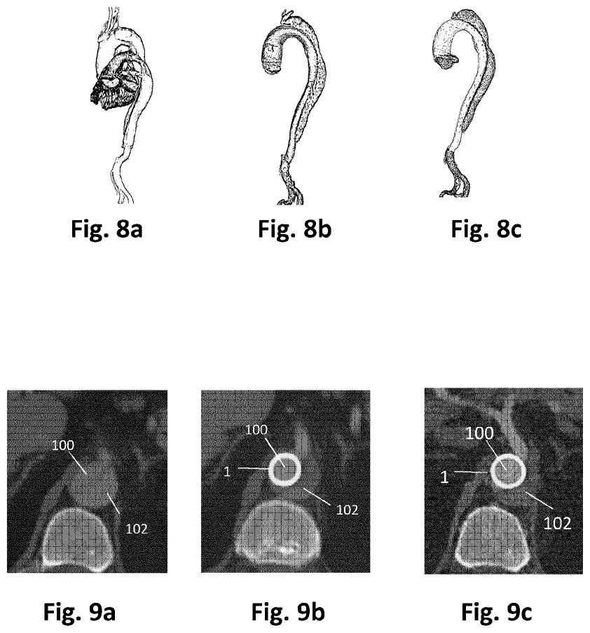

A case of complicated type B dissection was treated by implanting endovascular prostheses according to the present disclosure from the ascending aorta to the abdominal aorta. FIGS. 7a to 7c show computed tomography (CT) scans of this case sliced at the plane of maximum compression where maximum compression of the TL was observed preoperatively. At preoperative stage, the TL was collapsed by compression from the FL (FIGS. 7a and 8a). The celiac trunk was originating from both the true and false lumens. The left renal artery was being supplied by the false lumen, whereas the remaining aortic branches were arising from the true lumen (FIG. 7a). During an implanting procedure, the endovascular prostheses according to the present disclosure was deployed along the curve of aortic arch from the ascending aorta to the abdominal aorta into the TL while covering an entry tear and a re-entry tear of FL as well as orifices of aortic branches including the brachiocephalic trunk, left common carotid artery, left subclavian artery, intercostal arteries, lumbar arteries, celiac trunk, superior mesenteric artery, renal arteries. At postoperative evaluation, TL was increased through re-approximation of the intimal flap after deployment of the endovascular prosthesis with a concomitant decrease of the FL diameter(s) (FIGS. 7b and 8b). At 36-month Follow-up, the endovascular prothesis provided support to the TL resulting in progressive decrease of the size of FL while maintaining the blood flow into the branches (FIGS. 7c and 8c). In conclusion, the implantable endovascular prosthesis according to the present disclosure exhibited sufficient flexibility to conform with the curves of aorta as well as adequate mechanical properties for allowing re-approximation and support of the intimal flap, re-expanding the TL diameter and restoring blood flow into the TL. As a result, the endovascular prosthesis led to a successful remodeling of dissected aorta while keeping appropriate blood circulation into the aortic branches even when the orifices of those branches were covered by the prosthesis.

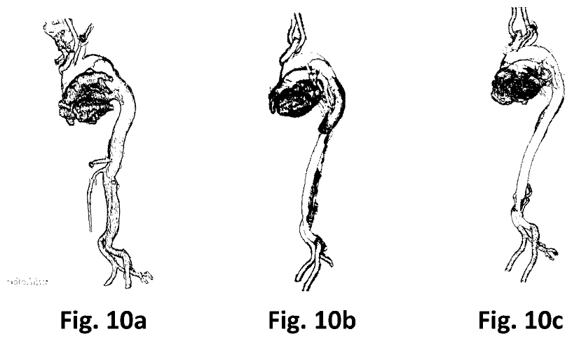

Another case of type B aortic dissection (FIG. 10a) was treated using endovascular prostheses according to the present disclosure. The endovascular prostheses were implanted from the left common carotid artery (aortic arch) to the aortoiliac bifurcation as shown in FIG. 10b (at discharge) and FIG. 10c (at 68-month follow-up). Computed tomography (CT) scans at the celiac trunk level were used to evaluate the condition before procedure, at discharge and at 68-month follow-up (FIGS. 9a to 9c, respectively). Prior to the intervention, the true lumen (TL) was compressed by the false lumen (FL), as observed in FIGS. 9a and 10a. The left renal artery originated from both the true and false lumens, while the remaining aortic branches arose from the TL. The endovascular prostheses according to the present disclosure were deployed from the aortic arch, just distal to left common carotid artery to the aortoiliac bifurcation into the TL, covering entire length of the dissection, as well as orifices of aortic branches including the left subclavian artery, intercostal arteries, lumbar arteries, celiac trunk, superior mesenteric artery, right and left renal arteries and inferior mesenteric artery (FIGS. 10b and 10c). Postoperative CT scan evaluation revealed an increase in TL diameter through reopening of the TL and a decrease in FL diameter (FIGS. 9b and 10b). At the 68-month follow-up, the endovascular prosthesis provided support to the TL, resulting in a progressive decrease in the size of the FL while maintaining blood flow into the aortic branches (FIGS. 9c and 10c). In conclusion, the implanted endovascular prosthesis according to the present disclosure demonstrated adequate flexibility, allowing it to conform to the natural curves of the aorta. Additionally, it displayed favorable mechanical properties, facilitating the reopening and support of the TL. This resulted in the successful remodeling of the dissected aorta, with a significant increase in the diameter of the TL and a subsequent decrease in the diameters of the FL. Furthermore, the endovascular prosthesis according to the present disclosure demonstrated its effectiveness in maintaining appropriate blood flow to the aortic branches even covered by the endoprosthesis.

TABLE 1
Repeating unit of braiding pattern of Ex.1 according to the present disclosure showing wires
positioning at outer side where crossing with wires in the opposite helical direction.

| | | | | | | | | | | | | | | | Number of | | |
|---|---|---|---|---|---|---|---|---|---|---|---|---|---|---|---|---|---|
| | | | | | | | | | | | | | | | times that A- | | |
| | | | | | | | | | | | | | | | wires cross at | | |
| | | Wires in clockwise rotation (C-wires) | | | | | | | | | | | | | outer side in a | | |
| | | C01 | C02 | C03 | C04 | C05 | C06 | C07 | C08 | C09 | C10 | C11 | C12 | C13 | repeating unit | | |
| Wires | A01 | C | C | A | C | C | C | C | C | A | C | C | C | C | 2 | 15.4% | IM |
| in | | | | | | | | | | | | | | | | | |
| anticlockwise | A02 | C | C | A | A | C | A | C | C | A | A | C | C | A | 6 | 46.2% | |
| rotation | | | | | | | | | | | | | | | | | |
| (A-wires) | A03 | A | C | A | A | A | A | A | C | A | A | A | A | A | 11 | 84.6% | OM |
| | A04 | C | C | C | C | C | A | C | C | C | C | C | C | A | 2 | 15.4% | IM |
| | A05 | A | C | A | C | C | A | A | C | A | C | C | C | A | 6 | 46.2% | |
| | A06 | A | A | A | A | C | A | A | A | A | A | C | C | A | 10 | 76.9% | |
| | A07 | C | C | A | C | C | C | C | C | A | C | C | C | C | 2 | 15.4% | IM |
| | A08 | C | C | A | A | C | A | C | C | A | A | C | C | A | 6 | 46.2% | |
| | A09 | A | C | A | A | A | A | A | C | A | A | A | A | A | 11 | 84.6% | OM |
| | A10 | C | C | C | C | C | A | C | C | C | C | C | A | A | 3 | 23.1% | |
| | A11 | C | C | C | C | C | A | C | C | C | C | C | C | A | 2 | 15.4% | IM |
| | A12 | A | C | A | C | C | A | A | C | A | C | C | C | A | 6 | 46.2% | |
| | A13 | A | A | A | A | C | A | A | A | A | A | C | C | A | 10 | 76.9% | |

| | 7 | 11 | 3 | 7 | 11 | 2 | 7 | 11 | 3 | 7 | 11 | 10 | 2 |
|---|---|---|---|---|---|---|---|---|---|---|---|---|---|
| Number of times | 53.8% | 84.6% | 23.1% | 53.8% | 84.6% | 15.4% | 53.8% | 84.6% | 23.1% | 53.8% | 84.6% | 76.9% | 15.4% |
| that C-wires cross | | OM | | | OM | IM | | OM | | | OM | | IM |

TABLE 2
Braiding pattern corresponding to FIG. 6a

| | C-i | C-ii | C-iii |
|---|---|---|---|
| A-i | A | C | A |
| A-ii | C | C | C |
| A-iii | A | C | A |

TABLE 3
The number of wires belonging to outmost layer, innermost layer
or middle layers in each helical direction and its total numbers

| | | Number of | Number of | Number of wires |
|---|---|---|---|---|
| | Total | wires defined | wires defined | belonging to |
| | number | as OM wires | as IM wires | middle layers |
| | of wires | (%) | (%) | (%) |
| Ex. 1 | 104 | 24 (23.1%) | 24 (23.1%) | 56 (53.8%) |
| Ex. 2 | 112 | 24 (21.4%) | 20 (17.9%) | 68 (60.7%) |
| Ex. 3 | 160 | 48 (30.0%) | 32 (20.0%) | 80 (50.0%) |
| CEx. 1 | 136 | 56 (41.2%) | 56 (41.2%) | 24 (17.6%) |
| CEx. 2 | 160 | 64 (40.0%) | 32 (20.0%) | 64 (40.0%) |

TABLE 4
The number of times that a wire crosses at outer
side with wires helically braided in the opposite
direction within a complete helical wrap

| | Total | | | | | |
|---|---|---|---|---|---|---|
| | number | Wire type | | | | |
| | of crossing | | | | | |
| | points | | | | | Wires |
| | per wire | | | | | belonging |
| | within a | | | | | to |
| | complete | OM wire | | IM wire | | middle |
| | helical wrap | C-wires | A-wires | C-wires | A-wires | layers |
| Ex. 1 | 52 | 44 | 44 | 8 | 8 | 12-40 |
| | | (84.6%) | (84.6%) | (15.4%) | (15.4%) | (23-77%) |
| Ex. 2 | 56 | 48 | 48 | 8 | 4 | 16-40 |
| | | (85.7%) | (85.7%) | (14.3%) | (7.1%) | (29-71%) |
| Ex. 3 | 80 | 56 | 72 | 8 | 16 | 16-48 |
| | | (70.0%) | (90.0%) | (10.0%) | (20.0%) | (20-60%) |
| CEx. 1 | 68 | 60 | 44 | 24 | 8 | 28-40 |
| | | (88.2%) | (64.7%) | (35.3%) | (11.8%) | (41-59%) |
| CEX. 2 | 80 | 64 | 56 | 8 | 16 | 16-48 |
| | | (80.0%) | (70.0%) | (10.0%) | (20.0%) | (20-60%) |

TABLE 5
Number of times that a repeating unit
is found within a complete helical wrap

| | | Number of wires | |
|---|---|---|---|
| | Total | per repeating | Number of |
| | number of | unit in a helical | repeating |
| | wires | direction | units per pitch |
| Ex. 1 | 104 | 13 | 4 |
| Ex. 2 | 112 | 14 | 4 |
| Ex. 3 | 160 | 10 | 8 |
| CEx.1 | 136 | 17 | 4 |
| CEx.2 | 160 | 10 | 8 |

TABLE 6
Measured thickness of braided framework and calculated T/D ratio

| | Thickness of a wall | | |
|---|---|---|---|
| | of braided | | |
| | framework | Wire Diameter | |
| | (T₂, mm) | (D₃, μm) | T₂/D₃ ratio |
| Ex. 1 | 0.72 | 190 | 3.79 |
| Ex. 2 | 0.79 | 200 | 3.95 |
| Ex. 3 | 1.01 | 210 | 4.81 |
| CEx. 1 | 1.07 | 210 | 5.10 |
| CEx. 2 | 1.03 | 210 | 4.90 |

TABLE 7
Crush resistance, elastic recovery and self-expandable property

| | Crush | Elastic | Self-expandable |
|---|---|---|---|
| | resistance force | recovery | property |
| | (N) | (%) | (Conformability) |
| Ex. 1 | 19.7 | 98.7% | Good |
| Ex. 2 | 24.9 | 97.5% | Good |
| Ex. 3 | 23.3 | 96.0% | Good |
| CEx. 1 | 31.2 | 87.5% | Poor |
| CEx. 2 | 22.1 | 93.3% | Poor |

1. An implantable endovascular prosthesis for use in a treatment of an aortic dissection comprising at least one self-expandable braided framework extending along an axis able to expand from a radially compressed state in a delivery configuration to a radially expanded state; the self-expandable braided framework being formed by wires of a same diameter, devoid of any impermeable cover layer, comprising a plurality of layers of wires made of biocompatible material, and forming a part of a wall of the implantable endovascular prosthesis; the plurality of layers of wires consisting of a lumen comprised in the self-expandable braided framework, forming a cylindrical shape with a circular cross-section; each layer forming a mesh; the meshes forming a lattice with a plurality of wires of said layers; the meshes being interlocked; and
the wires being integrated in the mesh of at least one of the adjacent layers; each wire forming a helical path in a direction or in the other around the axis of the self-expandable braided framework while crossing with wires of the opposite helical direction being positioned at either an outer or an inner side of the self-expandable braided framework; wherein a ratio of a thickness of a wall of said self-expandable braided framework to the diameter of the wires is at least 2.8, preferably at least 3.0, most preferably at least 3.5,
wherein wires taking position at the outer side the most often among wires of the same helical direction when crossing with wires of the opposite helical direction during a complete helical wrap are defined as wires belonging to the outermost layer of the self-expandable braided framework (OM wires); wires taking position at the outer side the least often among wires of the same helical direction when crossing with wires of the opposite helical direction during a complete helical wrap being defined as wires belonging to an innermost layer of the self-expandable braided framework (IM wires); wires not being OM wire nor IM wire being defined as wires belonging to middle layers of the self-expandable framework,
wherein the number of OM wires is at least 5% and at most 35% of the total number of wires forming of the self-expandable braided framework, the number of IM wires being at least 10% and at most 35% of the total number wires forming of the self-expandable braided framework.

2. The implantable endovascular prosthesis according to claim 1, wherein a pattern indicating positions of wires at crossing points is repeated at least 4 times within a complete helical wrap of the wires in both helical directions, preferably at least 8 times.

3. The implantable endovascular prosthesis according to claim 1, wherein the number of wires not being OM wire nor IM wire is at least 50% and at most 70% to the total number of wires forming the self-expandable braided framework.

4. The implantable endovascular prosthesis according to claim 1, wherein a ratio of the number of times that an OM wire crosses at outer side to the number of the total crosses made within its complete helical wrap is at least 70% and at most 90%.

5. The implantable endovascular prosthesis according to claim 1, wherein the number of wires forming the self-expandable braided framework is at least 72, preferably at least 96, more preferably at least 120 and 200, even more preferably between 150 and 180.

6. The implantable endovascular prosthesis according to claim 1, wherein a surface coverage ratio (SCR) of the self-expandable braided framework [(2)] in a fully expanded state is comprised between 25% and 45%, preferably between 30 and 40%.

7. The implantable endovascular prosthesis according to claim 1, wherein the wires forming the self-expandable braided framework have a diameter of at least 120 μm and at most 250 μm, preferably at least 150 μm and at most 220 μm, more preferably at least 180 μm and at most 210 μm.

8. The implantable endovascular prosthesis according to claim 1, wherein the cylindrical form of the self-expandable braided framework has a constant diameter in the fully expanded state.

9. The implantable endovascular prosthesis according to claim 8, wherein the self-expandable braided framework has an inner diameter of at least 20 mm and at most 50 mm in the fully expanded state.

10. The implantable endovascular prosthesis according to claim 1, wherein the self-expandable braided framework has larger diameter one or both end(s) in the fully expanded state.

11. The implantable endovascular prosthesis according to claim 10, wherein an inner diameter of the self-expandable braided framework is at least 20 mm and at most 50 mm in the fully expanded state.
